(19) United States
(12) Patent Application Publication
(10) Pub. No.: US 2021/0215654 A1
(43) Pub. Date: Jul. 15, 2021

(54) System and Method for Multidimensional Gas Sensing and Localization

(71) Applicant: Richard M. Costanzo, Manakin Sabot, VA (US)

(72) Inventor: Richard M. Costanzo, Manakin Sabot, VA (US)

(21) Appl. No.: 17/248,075

(22) Filed: Jan. 7, 2021

Related U.S. Application Data
(60) Provisional application No. 62/958,521, filed on Jan. 8, 2020.

(51) Int. Cl. G01N 33/00

(52) U.S. Cl. CPC G01N 33/0031

(57) ABSTRACT

The present invention provides a gas detection systems and methods to detect gases and gas concentrations in three-dimensional space. The present invention monitors the environment for gases and detects different gases, gas concentrations and localizes the direction of impinging gas molecules and their relative concentrations. The present invention consists of gas sensing elements on a 3-dimensional surface, electronic components, a processor and output signals. The output signals are used to determine the type of gas, it's concentration at different locations and changes in concentration that occur over time. The system can be used to generate gas distribution profiles (concentration maps) and movement of gases (velocity maps) for real-time gas monitoring and predicted changes.

### BACKGROUND OF THE INVENTION

### Field of the Invention

The present invention generally relates to gas sensors and gas sensing systems and, more particularly, to the detection and localization of gases and gas concentrations in three-dimensional space.

### Background Description

### SUMMARY OF THE INVENTION

Gas sensing arrays are distributed over a 3-Dimensional surface so as to optimize detection of gases impinging from different directions. The signal intensity from the gas arrays are used to determine the gas concentration or intensity. The orientation of the array on the surface and the intensity of the gas detected is used to generate directional vectors and to map the gas concentrations detected. Multiple gas sensing elements within a sensor, and the use of selectivity filtering for each element, allows for the generation of different response patterns or chemical fingerprints across the outputs of the array and is used to encode the chemical composition of the gas.

### BRIEF DESCRIPTION OF THE DRAWINGS

FIG. 1. Diagram illustrating the 3 components of the invention

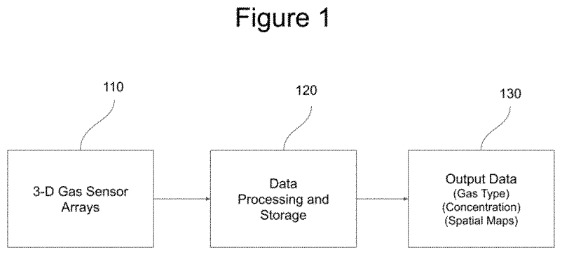

FIG. 2 Diagram illustrating the gas sensors and sensor arrays

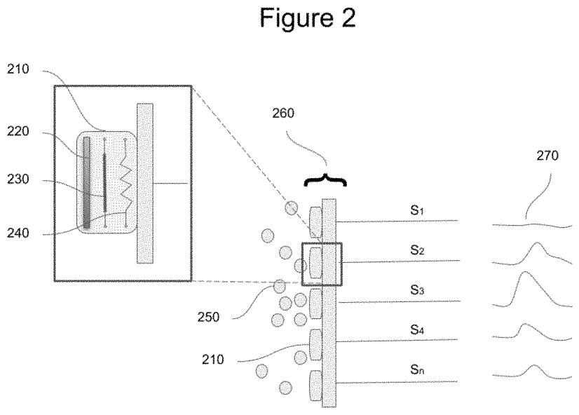

FIG. 3. Diagram illustrating the gas sensor arrays on 3-dimensional structures

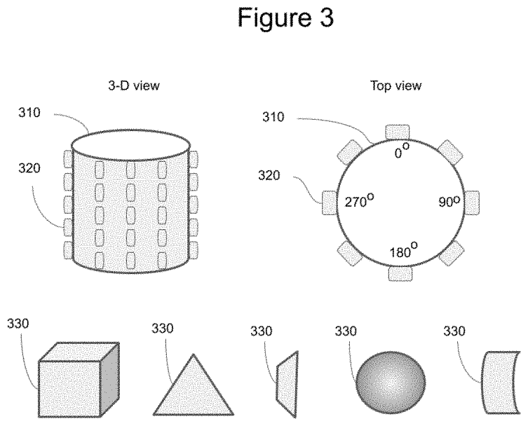

FIG. 4. Diagram illustrating the processing of the gas sensor information

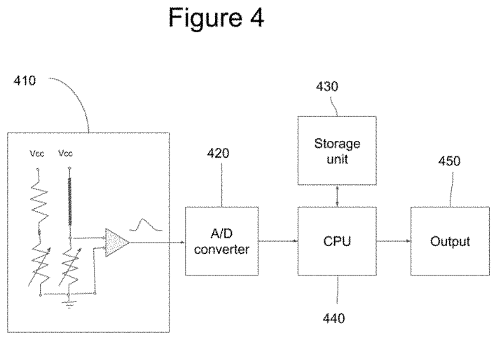

FIG. 5. Diagram illustrating relative position of a gas source and gas intensity vectors

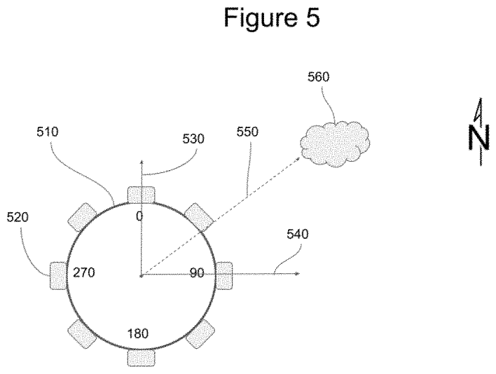

FIG. 6. Diagram illustrating deployment of multiple gas sensors and gas intensity plots

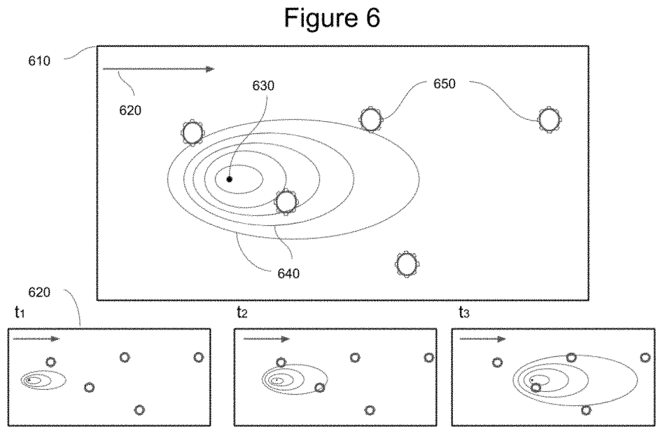

FIG. 7. Listing of steps in the process of using 3-D sensing systems

FIG. 8. Photograph of a working 3-D gas sensing system prototype

FIG. 9. Data from the gas sensing prototype shown in FIG. 8

### DETAILED DESCRIPTION

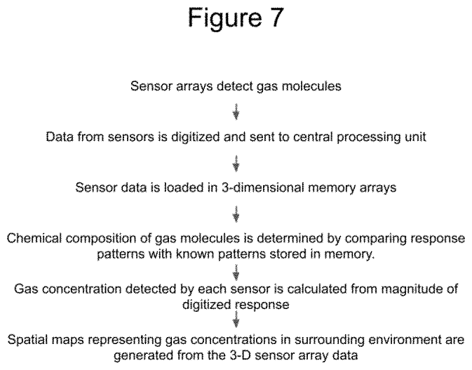

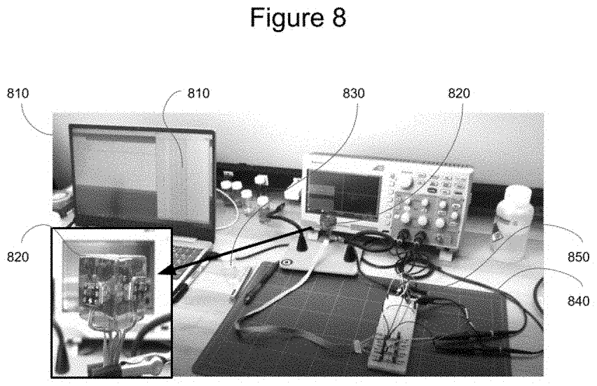

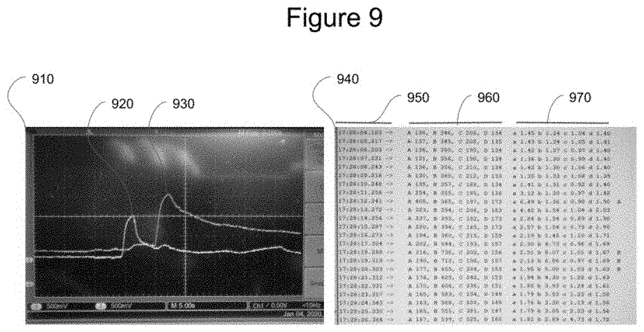

This invention enables new gas sensing methods and applications that can be deployed with handheld units, remote sensing units, cell phones, drones and other systems. The invention consists of 3 components: gas sensors located on 3-Dimensional surfaces [110], electronics that include a central processor, memory [120], and output signals that encode the type of gas (odor), its concentration and spatial maps [130]. The gas sensor arrays consists of individual gas sensors [210] comprised of a sensing element [230], and heating element [240] and a selectivity filter [220] that is differentially tuned so that across sensors a unique response pattern is generated [270] for different gas molecules (odors). Increasing the number of sensors and surface area of the sensor array increases the sensitivity and selectivity. The arrangement of sensors on 3-D surfaces [330] allows for improved gas detection since chemicals in the vapor phase typically surround the sensor and most commercial sensors are designed for detection at one surface and dimension. With multiple arrays of sensors distributed over the surface [320] and oriented in different directions gas concentrations can be measured and predicted for positions between sensors. The sensor conditioning electronics [410] generates analog signals [270] that are converted to digital signals [420] and sent to a central processor [440] for storage [430] and analysis. Signals from the sensor arrays are stored in corresponding memory 3-D matrix arrays and represent a gas profile that includes unique patterns or digital fingerprints that identify the specific odorant gas in addition to concentration data. The sensor array data (odor fingerprint) stored in memory is compared to known patterns pre-programmed or obtained by exposing the sensor arrays to known calibrated gases. Output data from the central processor [450] includes lists and graphic plots that contain the identity and concentration of gas vapors as well as spatial maps or plots of the gas concentration in 3-dimensional space. The relative intensity or concentration at different sensors can be represented by vectors [530,540] and used to predict intensity vectors that project onto different locations [550]. In addition deployment of multiple 3-D sensors [650]in an environment [610] can be used to obtain information that maps the concentration topography [640] around a gas source [630] as well as the changes in concentration topography maps [620] with changes in time (t1,t2,t3). The method or process of using a multidimensional sensor array system is illustrated in FIG. 7. FIG. 8 shows a working prototype system[810]that employs sensors on a 4 sided cube [820]. The prototype included a laptop computer programmed to run a small microprocessor [850] that supplied power to the sensors mounted on the cube [820], received analog data from the 4 sensor output circuits [840], digitized the voltage signals and displayed them on the laptop computer [810]. Sample data is displayed in FIG. 9. The sensor voltage changes are displayed on an oscilloscope screen [910] and represent the responses to isopropyl alcohol presented to sensor A [920] and then to sensor B [930]. The computer readout from the microprocessor [940] shows a time stamp [950], the A/D digitized response data for sensors A, B, C and D [960] and the relative concentrations detected for each on the sensors [970]. Information obtained using 3-D surface sensor arrays enables multiple applications. Gas vapor detection has importance for food safety, industrial manufacturing, environmental monitoring, homeland security, military equipment, and more.

The application of multiple gas sensors on 3 dimensional surfaces represents a novel approach to advance current gas sensing technologies. Miniaturization allows for high density packing of sensors to form sensor arrays. Surface modifications and filtering techniques allows for improved selectivity of sensory arrays. Modifications and variations in surface coating of exposed surface areas of the sensors accomplished by using organic and inorganic materials (e.g. charcoal doping) to produce differential responses from individual sensors in the array.

One advantage of a 3D gas sensor array is that it can help to “localize” the point of origin or location of a gas being released into the environment. Using data based on changes in signal intensity across the surface of the array it is possible to detect directional movement of a gas. In addition the deployment of multiple sensors positioned at different locations allows for the generation of concentration mapping as well as changes that occur over time (changes in gas mapping and velocity and direction of movement). Both commercial and military applications are envisioned. The system would be useful in detecting not only the location of a potential environmental threat but also if the threat is changing (getting closer, moving to the left or right, or holding constant).

Having thus described the invention, what is claimed as new and desired to secure by patent is as follows:

1. A method for detecting gas molecules and relative changes in gas concentrations in 3-dimensional space, and generating output data that identifies the type of gas molecules, the relative changes in gas concentrations in 3-dimensional space and spatial mapping of the presence and movement of gases.

2. A system for detecting and mapping gas molecules in 3-dimensional space.

3. The method as claimed in claim 1, wherein an array of sensors is used to identify gas molecules by analyzing response patterns across selectively tuned sensors so as to generate unique patterns, or chemical fingerprints, for gases with different chemical compositions.

4. The method as claimed in claim 1, wherein gas sensors arranged at different locations on the surface of 3-dimensional structures is used to detect the relative concentrations of gases.

5. The method as claimed in claim 1, wherein gas sensors are arranged on the surface of 3-dimensional structures that rotate or move to obtain data on the concentrations of gases.

6. The method as claimed in claim 1, wherein gas sensors arrays arranged at different locations on the surface of 3-dimensional structures to detect the relative concentrations of gases in 3-dimensional space.

7. The method as claimed in claim 1, where multiple 3-D structures containing arrays of gas sensors are deployed in the environment so as to monitor and map changes in the distribution of gas concentrations over time.

8. The system as claimed in claim 2, comprised of gas sensing arrays, where each gas sensing element in the array has a filter that allows for a differential response to the presence of different gas molecules.

9. The system as claimed in claim 2, wherein a central processor is used to compare response patterns across the sensor array outputs to determine the type of gas by comparing patterns to those programmed and stored in a memory unit.

10. The system as claimed in claim 2, that includes output devices that display sensor data including the gas type, gas concentrations, and maps showing the distribution of gas concentrations throughout the sensing environment.
